(19) United States
(12) Patent Application Publication
(10) Pub. No.: US 2009/0068181 A1
(43) Pub. Date: Mar. 12, 2009

(54) BISPECIFIC ANTIBODIES

(75) Inventors: Randall J. LEE, Hillsborough, CA (US); James Larrick, Mountain View, CA (US); Lawrence G. Lum, Coventry, RI (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: TransTarget, Inc., Hillsborough, CA (US)

(21) Appl. No.: 12/207,285

(22) Filed: Sep. 9, 2008

Related U.S. Application Data
(63) Continuation of application No. 11/170,716, filed on Jun. 28, 2005.
(60) Provisional application No. 60/583,946, filed on Jun. 28, 2004.

(51) Int. Cl. A61K 39/395; A61K 39/44; C07K 16/28; C12N 5/06

(52) U.S. Cl. 424/133.1; 424/172.1; 424/178.1; 424/173.1; 530/389.1; 435/325; 530/389.6

(57) ABSTRACT

The present invention provides compositions and methods for targeting stem cells to injured cardiac tissue.

### CROSS-REFERENCES TO RELATED APPLICATIONS

This application claims the benefit of U.S. Provisional Patent Application No. 60/583,946, filed Jun. 28, 2004, the disclosure of which is incorporated by reference in its entirety for all purposes.

### BACKGROUND OF THE INVENTION

Cardiovascular disease (CVD) is the leading cause of death in the US with an estimated 60 million patients costing the healthcare system approximately $186 billion annually (Lenfant, Circulation, 95:771-772 (1997); Cohn et al., Circulation, 95:766-770 (1997)). A large proportion of CVD results from ischemic heart disease. Currently, heart transplantation is the only successful treatment for end-stage heart failure; however, the ability to provide this treatment is limited by the availability of donor hearts (el Oakley et al., J Heart Lung Transplant., 15:255-259 (1996); Keck et al., Worldwide thoracic organ transplantation: a report from the UNOS/ISHLT International Registry for Thoracic Organ Transplantation. In: Clinical Transplants. (1999)). Therefore, alternative therapies are needed to treat end-stage heart failure. Recently, significant advances in cellular transplantation has gained enthusiasm as an alternative treatment for myocardial repair.

Cell transplantation and cardiac tissue engineering show promise for repairing damaged myocardium. The basic issues common to all cell types used for cardiac repair are identification, survival of engrafted cells, differentiation, host tissue-transplant cell interactions, and electromechanical coupling (Klug et al., J Clin Invest., 98:216-224 (1996)). Investigators have successfully transplanted fetal and neonatal cardiac myocyte suspensions into the myocardium (Hamill et al., Pflugers Arch., 391:85-100 (1981); Li et al., Ann Thorac Surg., 62:654-660 (1996); Mar et al., Circulation, 96(I), 556 (1997); Scorsin et al., Circulation, 96:11-93 (1997)). Transplantation of fetal cardiac myocytes improved emodynamics (Klug et al., J Clin Invest., 98:216-224 (1996)). The inherent electrophysiologic, structural, and contractile properties of fetal cardiac myocytes enabling functional integration into host myocardium suggest that fetal cardiac myocytes can repair myocardial injury (Atkins et al., J Surg Res., 85:234-242 (1999)).

Non-fetally-derived cells have also been investigated including, e.g., peripheral blood stem cells (PBSC), stem cells isolated from bone marrow; stem cells isolated from adipose tissue; mesenchymal stem cells, and CD34⁺ cells. For example, it has been shown that bone marrow derived stem cells injected directly into the myocardium shortly after coronary ligation can repair injured myocardium by producing myocytes and vascular structures in the infarcted portion (see, e.g., Orlic et al., Nature, 410:701-705 (2001)). Furthermore, homing, replication, differentiation, and repair of injured myocardium was enhanced by granulocyte-colony-stimulating factor (G-CSF) which mobilize SC from the BM (Orlic et al., PNAS USA, 98:10344-10349 (2001) and Orlic et al., Blood, 82:762-770 (1993)). In humans, mobilized SC have been characterized as either CD34⁺ or CD34⁻ populations.

Delivery of stem cells to the myocardium is critical in determining the efficacy of the cell therapy. Both intracoronary delivery and intravenous delivery of stem cells have demonstrated feasibility of transplantation of autologous bone marrow mononuclear cells in patients with ischemic myocardial injury (Strauer et al., Circulation, 106:1913-1918 (2002); Tse et al., Lancet, 361:47-49 (2003)). Several clinical studies have demonstrated the safety and feasibility of intracoronary or intramyocardial transplantation of autologous bone marrow mononuclear cells (BMC) to induce myocardial regeneration and neovascularization in patients with ischemic myocardial injury (Strauer et al., Circulation, 106:1913-1918 (2002); Tse et al., Lancet, 361:47-49 (2003); Perin et al., Circulation, 107:2294-2302 (2003)). In addition, recent MR studies have demonstrated the survival of intramyocardially injected stem cells 4-6 weeks after administration (Dick et al., Circulation, 108:2899-2904 (2003)).

Proponents of direct intramyocardial injection suggests that ischemic myocardium does not have the necessary circulation to allow stem cells to reach injured myocardium. Proponents of intracoronary delivery of stem cells claim that intramyocardial injection of stem cells is limited to the site of injection, thus requiring multiple injections to the infarct area. Additionally, intramyocardial injections are not restricted to the infarct region and cell retention is limited. Serious concerns have been raised regarding the risk of life-threatening ventricular arrhythmias resulting from direct bolus injections of SC into the heart. Thus, additional studies to identify non-invasive approaches that increase therapeutic efficacy while minimizing risk of LV catheterization and intramyocardial intervention are needed.

One approach for targeting stem cells to injured myocardium using a chemically conjugated bispecific antibody to treat cardiovascular disease has recently been developed (Lum et al., Blood Cells Mol Dis., 32(1):82-7 (2004)). The bispecific antibody consists of two Fab fragments, one that specifically binds to the tyrosine kinase receptor, c-kit, and one that specifically binds to the cellular adhesion molecule, VCAM-1. Purified Lin⁻Sca⁺ murine stem cells were armed with the bispecific antibody and were wither directly injected into the myocardium of mice with infarcts created by ligation of the left anterior descending artery (LAD) were directly injected with armed stem cells, or injected via the internal jugular vein of such mice. Since both of the Fab fragments specifically bind to proteins that are widely expressed on multiple tissue types, however, the bispecific antibody described by Lum et al. lacks the specificity needed to efficiently target stem cells to injured myocardium.

Thus, there is a need in the art for improved methods of specifically targeting stem cells to injured cardiac tissue (e.g., myocardium). The present invention addresses these and other needs.

### SUMMARY OF THE INVENTION

The present invention provides compositions and methods for targeting cells (e.g., stem cells) to injured cardiac tissue (e.g., myocardium).

One embodiment of the invention provides a composition comprising a polypeptide comprising a cardiac antigen-specific binding component and a stem cell antigen-specific binding component. The cardiac antigen-specific binding component specifically binds to a cardiac-specific antigen (e.g., myosin light chain or troponin I) available for binding to the cardiac antigen-specific binding component and the stem cell antigen-specific binding component specifically binds to an antigen expressed on the surface of a stem cell (e.g., a peripheral blood stem cell (PBSC), a stem cell isolated from bone marrow; a stem cell isolated from adipose tissue; a mesenchymal stem cell, a CD34⁺ cell, a CD34⁻ cell, or combinations thereof). The cardiac antigen-specific binding component and the stem cell antigen-specific binding component may be chemically conjugated. In some embodiments, the polypeptide is bound to a stem cell via the stem cell antigen-specific binding component. The cardiac antigen-specific binding component and the stem cell antigen-specific binding component may be antibodies including intact antibodies and antibody fragments such as, e.g., (Fab)′2 fragments, Fab fragments, scFv; modified antibodies such as, e.g., humanized antibodies; or antibody mimetics such as, e.g., anticalins. In some embodiments, the stem cell antigen-specific binding component specifically binds to CD9, CD29, CD34, CD44, CD45, CD49e, CD54, CD71, CD90, CD105, CD106, CD120a, CD124, CD166, Sca-1, SH2, or SH3.

Another embodiment of the invention provides a method for targeting stem cells to injured cardiac tissue by administering to a subject (e.g. a mammal, including a rodent or a primate such as a human) a composition comprising a polypeptide bound to a stem cell. The polypeptide comprises a cardiac antigen-specific binding component that specifically binds to a cardiac-specific antigen (e.g., myosin light chain or troponin I) available for binding to the first component and a stem cell antigen-specific binding component that specifically binds to an antigen expressed on the surface of the stem cell (e.g., a peripheral blood stem cell (PBSC), a stem cell isolated from bone marrow; a stem cell isolated from adipose tissue; a mesenchymal stem cell, a stem cell isolated from umbilical cord blood, a CD34⁺ cell, a CD34⁻ cell, or combinations thereof). In some embodiments, the stem cell antigen-specific binding component specifically binds to CD9, CD29, CD34, CD44, CD45, CD49e, CD54, CD71, CD90, CD105, CD106, CD120a, CD124, CD166, Sca-1, SH2, SH3, or HLA Class I.

A further embodiment of the invention provides the use, in the manufacture of a medicament for targeting stem cells to injured cardiac tissue of the polypeptides described herein.

These and other embodiments of the invention are described in greater detail below.

### BRIEF DESCRIPTION OF THE DRAWINGS

FIG. 1A illustrates chemical conjugation to generate a bispecific antibody the specifically binds to both CD45 and myosin light chain (MLC). FIG. 1B illustrates data from flow cytometry analysis of peripheral blood stem cells alone or bound to a bispecific antibody that specifically binds to both CD45 and MLC. The inset shows Coomassie blue staining of an acrylamide gel of the conjugation products.

FIG. 2 illustrates data comparing sequential echocardiography results from rats that received infusions of CD34+ cells either armed or unarmed with CD45xMLC. Bars indicate the SEM; n=9 armed rats and 8 unarmed rats; (**) p<0.01; (*) p<0.05

### DETAILED DESCRIPTION OF THE INVENTION

The present invention is based on the discovery that stems cells armed polypeptides that specifically bind to (1) an antigen on the surface of the stem cell; and (2) a cardiac cell antigen (i.e., myosin light chain or troponin I) will preferentially home to injured cardiac tissue (e.g., injured myocardium) rather than normal cardiac tissue. These armed stem cells remain in the injured cardiac tissue where they can differentiate and facilitate the repair and healing of the injured cardiac tissue.

The polypeptides of the invention comprise at least two binding components: a cardiac antigen-specific binding component that specifically binds to myosin light chain or troponin I; and a stem cell antigen-specific binding component that specifically binds to an antigen expressed on the surface of a stem cell (e.g., CD9, CD29, CD34, CD44, CD45, CD49e, CD54, CD71, CD90, CD105, CD106, CD120a, CD124, CD166, Sea-1, SH2, SH3, and HLA Class I). In some embodiments, a composition comprising the polypeptides is administered to a patient with cardiovascular disease. The polypeptide homes to injured cardiac tissue and recruits stem cells (i.e., endogenous stem cells or exogenously administered stem cells) to the tissue. In other embodiments, a composition comprising such a polypeptide bound to a stem cell (i.e., an “armed stem cell”) is administered to a patient with cardiovascular disease. The armed stem cell homes to injured cardiac tissue. Targeting stem cells to injured myocardium using the polypeptides of the invention increases the number of stem cells migrating to injured cardiac tissue and increases the number of surviving, differentiated cells, thus leading to repair of the myocardium and a physiologic improvement of heart function in a patient with cardiovascular disease.

### I. DEFINITIONS

As used herein, the following terms have the meanings ascribed to them below unless otherwise specified.

The term “cardiac antigen-specific binding component” as used herein refers to a polypeptide (e.g., an antibody or an antibody mimetic such as, e.g., anticalin) that specifically binds to a cardiac antigen. As used herein, the term “cardiac antigen” refers to an antigen present in cardiac tissue (e.g., myocardium). A cardiac specific antigen may be an antigen expressed on the surface of cardiac cells or may be an antigen that is not on the surface of uninjured cardiac cells, but is exposed after an injury to cardiac tissue (e.g., ischemic injury or another injury induced by a lack of oxygen to cardiac tissue). For example, injuries to the myocardium caused by ischemic heart disease can lead to exposure of cardiac antigens such as myosin light chain and troponin I making them available for binding by a cardiac antigen-specific binding component. The cardiac antigens are expressed in injured cardiac tissue, but not in other types of tissue.

There are two types of myosin light chain: a regulatory light chain (i.e., MYL2) and an essential light chain (i.e., MYL3) (see, e.g., Yamashita et al., Cardiovascular Res. 60: 580-588 (2003)). The light chains stabilize the long alpha helical neck of the myosin head. The cardiac isoform of the myosin essential light chain is a 196 amino acid protein with a molecule weight of 21.865 kD. The cardiac isoform of the myosin regulatory light chain is a 163 amino acid protein with a molecule weight of 18.603 kD. Human myosin essential light chain sequences are set forth in, e.g., Genbank Accession Nos.: NM_{—}000258, CR456963, BC012571, BC009790, and M24122. A rat myosin essential light chain sequence is set forth in, e.g., Genbank Accession No.: NM_{—}012606. Human myosin regulatory light chain sequences are set forth in, e.g., Genbank Accession Nos. BC032748; BC031972; BC016372; BC004994; NM_{—}006471; NM_{—}033546. Rat myosin regulatory light chain sequences are set forth in, e.g., Genbank Accession Nos. BC060577 and NM_{—}017343. Additional myosin light chain sequences are set forth in, e.g., Genbank Accession Nos. BC012425 (human) and X51531 (rat).

Troponin I (TNN13 or TnI) is one of 3 subunits that form the troponin complex (i.e., a complex of TnI, TnT, and TnC) of the thin filaments of striated muscle, including cardiac muscle. The troponin complex plays a role in regulating cardiac muscle contraction. Human troponin I sequences are set forth in, e.g., Genbank Accession Nos.: NM_{—}000363×90780; and M64247) A rat troponin I sequence is set forth in, e.g., Genbank Accession No.: NM_{—}017144).

The term “stem cell antigen-specific binding component” as used herein refers to a polypeptide (e.g., an antibody or an antibody mimetic such as, e.g., anticalin) that specifically binds to an antigen that is expressed on the surface of a stem cell. Stem cells are pluripotent or multipotent cells that can differentiate into multiple cell types. Stem cells also include cells that can transdifferentiate into at least one other cell type. Stem cells include, e.g., peripheral blood stem cells (PBSC), stem cells isolated from bone marrow; stem cells isolated from adipose tissue; mesenchymal stem cells, stem cells isolated from umbilical cord blood, embryonic stem cells, CD34⁺ cells, CD34⁻ cells, CD9⁺ cells, CD29⁺ cells, CD44⁺ cells, CD45⁺ cells, CD49e⁺ cells, CD54⁺ cells, CD71⁺ cells, CD90⁺ cells, CD105⁺ cells, CD106⁺ cells, CD120a⁺ cells, CD124⁺ cells, CD166⁺ cells, Sca-1⁺ cells, SH2⁺ cells, SH3⁺ cells, and HLA Class I cells.

The term “antigen expressed on the surface of a stem cell” refers to a protein, carbohydrate, or glycoprotein present on the surface of a stem cell. Antigens expressed on the surface of a stem cell include antigens expressed solely on the surface of a stem cell as well as antigens expressed on other cells. Different types of stem cells express different cell surface markers and therefore cells can be identified by the presence of a cell surface marker. For example, stem cells may express CD9, (Genbank Accession No.: BC011988), CD29, (Genbank Accession Nos.: BC020057; NM_{—}133376: NM_{—}033669; NM_{—}033668; NM_{—}033667; NM_{—}033666; and NM_{—}002211), CD34 (Genbank Accession No.: BC039146), CD44 (Genbank Accession No.: NM_{—}001001392; NM_{—}001001391; NM_{—}001001390; NM_{—}001001389; NM_{—}000610), CD45 (Genbank Accession Nos.: NM_{—}002838; NM_{—}080921; NM_{—}080922; NM_{—}080923), CD49e (Genbank Accession Nos.: BC008786 and NM_{—}002205), CD54 (Genbank Accession No.: BC015969), CD71 (Genbank Accession Nos. BC001188; BX537966; M11507; and X01060), CD90, (Genbank Accession Nos.: BC065559 and NM_{—}006288), CD105, (Genbank Accession Nos.: AF035753; U37439; BC014271; BC020391; BT006872; J05481; X72012), CD106 (Genbank Accession Nos.: NM_{—}080682 and NM_{—}001078), CD120a (Genbank Accession No.: NM_{—}001065), CD124 (Genbank Accession Nos.: AC004525 and X52425), CD166 (Genbank Accession No.: NM_{—}001627), Sca-1 (Genbank Accession Nos.: BC002070 and NM_{—}010738), SH2 (Genbank Accession No.: NM_{—}207372), and SH3 (Genbank Accession No.: BC069511), HLA Class I (Genbank Accession Nos. S42062; S42047; and NM_{—}005514) or combinations thereof.

The term “antibody” refers to a polypeptide encoded by an immunoglobulin gene or functional fragments thereof that specifically binds and recognizes an antigen (e.g., a cardiac antigen such as myosin light chain or troponin I, or a stem cell antigen). Myosin light chain specific monoclonal antibodies include, e.g., MLM508 and MLM544 (Abeam Ltd., Cambridge, Mass.); MAB150 and MAB160 (Accurate Chemical & Scientific Corporation, Westbury, N.Y.); 8-3 F6 and F5 (ERFA, Canada). Troponin I specific monoclonal antibodies include, e.g., 8E10, 414, B2, C5, 4C2, 19C7, 16A11, 18H7, 10B11, 3C7, 23C6, 7F4, 16A12, P4-14G5, P4-3A5, M18, M155, M46, MF4, and 3G1 (Advanced Immunochemical Inc., Long Beach, Calif.) and P4-3A5; P4-14G5; M18; 23C6; (280)4C2; 3C7; M155; (284)19C7; 8E10; (285)16A11; 16A12; 18H7 M46; 3G1; MF4; P420 (BIODESIGN International, Saco, Me.). CD45-specific monoclonal antibodies include, e.g., CLB-T200/1, 15D9; F10-89-4; RVS-1; and MEM28 (Research Diagnostics Inc, Flanders, N.J.) and UCHL1 (Zymed Laboratories, Inc., South San Francisco, Calif.); ML2, MT2, MT4, MB1 (IQ Products, Netherlands); LT45.M5 (Yorkshire Biosciences, Heslington, York); F8-11-13, H130, CLB-11G8 (Sanquin, Netherlands), Anti-HLe-1 (Becton Dickinson), and 2H4 (Beckman Coulter).

The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

“Antibody mimetics” are polypeptides comprising a binding component that specifically binds to an antigen (e.g., a cardiac antigen or a stem cell antigen as described herein). Antibody mimetics are typically polypeptides with protein scaffolds comprising one or more regions which are amenable to specific or random sequence variation such that the polypeptide specifically binds to an antigen of interest (e.g., a cardiac antigen or a stem cell antigen) and include, e.g., anticalins which are based on lipocalins and are described in Weiss and Lowman, Chem. Biol., 7(8)R177-84 (2000); Skerra, J. Biotechnol. 74(4):257-75; and WO99/16873; polypeptides with a fibronectin type III domain as described in, e.g. WO 01/64942 and polypeptides with a P sandwich structure as described in, e.g., WO 00/60070. The antibody mimetic may comprise the cardiac antigen-specific binding component, the stem cell antigen-specific binding component, or both. The antibody mimetic may be linked (i.e., conjugated) to another antibody mimetic or to an antibody via any means known in the art.

The term “linked” or “conjugated” in the context of the binding components of the present invention refers to the linkage between the cardiac antigen-specific binding component and the stem cell antigen-specific binding component. The linkage may be introduced through recombinant means or chemical means. Suitable methods for chemically linking two antibodies are described in, e.g., Sen et al., J. Hematother. Stem Cell Res. 2001 April; 10(2):247-60 (2001). Additional linkers and methods of linking antibody fragments such as scFv and dsFv are described in WO 98/41641. Additional exemplary chemical linkages include, for example, covalent bonding, including disulfide bonding; hydrogen bonding; electrostatic bonding; recombinant fusion; and conformational bonding.

The terms “effective amount” or “amount effective to” or “therapeutically effective amount” refers to an amount sufficient to induce a detectable therapeutic response in the subject. Preferably, the therapeutic response is effective in repairing injured cardiac tissue present in a subject. Assays for determining therapeutic responses are well known in the art. For example repair (i.e., healing) of injured myocardium can be detected using magnetic resonance imaging (MRI) to detect changes in the myocardium that are indicative of tissue regrowth and reformation.

### II. COMPOSITIONS OF THE INVENTION

One embodiment of the present invention provides a polypeptide comprising two binding components: a cardiac antigen-specific binding component that specifically binds to myosin light chain or troponin I and a stem cell antigen-specific binding component that specifically binds to an antigen expressed on a stem cell. These bispecific polypeptides can conveniently be used to “arm” stem cells and target them to injured myocardium. Once the armed stem cells have homed to the injured myocardium, they can differentiate into myocardial cells and facilitate repair of injured or diseased myocardium.

A. Bispecific Binding Molecule

The bispecific polypeptides of the invention typically comprise a bispecific antibody or antibody mimetic with a cardiac antigen binding component and a stem cell antigen binding component. Binding molecules that specifically bind to cardiac antigens and stem cells antigens can be generated using methods known in the art. For example, antibodies and antibody mimetics that specifically bind myosin light chain, troponin I, or CD45 may be generated for the bispecific binding molecules of the invention.

1. Antibodies

Methods of producing monoclonal antibodies that react specifically with cardiac antigens and stem cells antigens are known to those of skill in the art. For example, preparation of and monoclonal antibodies by immunizing mice with an appropriate immunogen is described in, e.g., Coligan, Current Protocols in Immunology (1991); Harlow & Lane, supra; Goding, Monoclonal Antibodies: Principles and Practice (2d ed. 1986); and Kohler & Milstein, Nature 256:495497 (1975). Antibody preparation by selection of antibodies from libraries of nucleic acids encoding recombinant antibodies packaged in phage or similar vectors is described in, e.g., Huse et al., Science 246:1275-1281 (1989) and Ward et al., Nature 341:544-546 (1989). In addition, antibodies can be produced recombinantly using methods known in the art and described in, e.g., Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994)).

a) Antigens

A number of antigens including, e.g., intact cardiac antigens, intact stem cells antigens, or portions of such antigens, can conveniently be used to produce antibodies that specifically bind to cardiac antigens and stem cell antigens. The antigens may be isolated from natural sources or may be recombinantly produced. For example, recombinant myosin light chain, troponin I, or CD45 sequences can be expressed in eukaryotic or prokaryotic cells and purified using methods known in the art. In some cases, the antigens can be purchased from commercial sources. For example, recombinant myosin light chain and troponin I can be purchased from multiple sources including, e.g., Abcam, Inc, Cambridge, Mass.; BiosPacific, Emeryville, Calif.; and Spectral Diagnostics Inc., Toronto, Ontario, Canada; and recombinant CD45 can be purchased from, e.g., CalBiochem, San Diego, Calif. and Biomol Research Laboratories Inc., Plymouth Meeting, Pa. The antigens can also be isolated from natural sources or produced recombinantly using methods known in the art. The antigens may be administered alone, in combination with an adjuvant (e.g. Freund's adjuvant), or conjugated to a carrier protein (e.g., KLH).

b) Antibody Production

The production of monoclonal antibodies is well known in the art. In general, spleen cells from an animal immunized with the desired immunogen (i.e., a myosin light chain, troponin I, or a stem cell antigen such as CD45) are immortalized, commonly by fusion with a mycloma cell (see, Kohler & Milstein, Eur. J. Immunol. 6:511-519 (1976) and Harlow & Lane, ANTIBODIES, A LABORATORY MANUAL, Cold Spring Harbor Publication, New York (1988)), transformation with Epstein Barr Virus, oncogenes, or retroviruses, or other methods well known in the art. Colonies arising from single immortalized cells are screened for production of antibodies of the desired binding specificity and binding affinity for the antigen.

Once a hybridoma that produces a monoclonal antibody that specifically binds to a myosin light chain, troponin I, or a stem cell antigen has been generated, the genes encoding the heavy and light chains can be cloned from the hybridoma cell that produces the monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3^{rd} ed. 1997)). Nucleic acids encoding antibodies that specifically bind to cardiac antigens, stem cell antigens, or portions thereof can be isolated directly from mRNA, from cDNA, or DNA libraries using methods such as polymerase chain reaction (PCR) and ligase chain reaction (LCR) (see, e.g., U.S. Pat. Nos. 4,683,195 and 4,683,202; PCR Protocols: A Guide to Methods and Applications (Innis et al., eds, 1990)). Phage display technology can be used to identify antibodies and Fab fragments that specifically bind to selected antigens (see, e.g., McCafferty et al., Nature 348:552-554 (1990); Marks et al., Biotechnology 10:779-783 (1992)). In addition, techniques for the production of single chain antibodies or recombinant antibodies (U.S. Pat. No. 4,946,778, U.S. Pat. No. 4,816,567) can be adapted to produce bispecific antibodies that specifically bind to cardiac antigens and stem cell antigens.

In addition to the antibodies generated using the methods described herein, multiple myosin light chain specific antibodies, troponin I specific antibodies, and stem cell antigen specific antibodies can be purchased from multiple sources (e.g., Abcam Ltd., Cambridge; Accurate Chemical & Scientific Corporation, Westbury, N.Y.; ERFA, Canada; Advanced Immunochemical Inc., Long Beach, Calif.; BIODESIGN International, Saco, Me.; Research Diagnostics Inc, Flanders, N.J.; IQ Products, Netherlands; Yorkshire Biosciences, Heslington, York; Sanquin, Netherlands; and Beckman Coulter) and used to generate the bispecific binding molecules described herein.

c) Immunoassays

Immunoassays known in the art can be used to assess the binding specificity, binding affinity, and epitope specificity of antibodies that specifically bind to cardiac antigens (e.g., myosin light chain or troponin I) or stem cell antigens (e.g., CD9, CD29, CD34, CD44, CD45, CD49e, CD54, CD71, CD90, CD105, CD106, CD120a, CD124, CD166, Sca-1, SH2, or SH3). Specific monoclonal antibodies will usually bind with a K_{A} of at least about 10⁻⁸, more usually at least about 10⁻¹⁰; and most preferably, about 10⁻¹² or better. For epitope mapping, a sterically competitive immunoassay can be used. For a review of suitable immunological and immunoassay procedures, see, e.g., Harlow & Lane, ANTIBODIES, A LABORATORY MANUAL, Cold Spring Harbor Publication, New York (1988); Basic and Clinical Immunology (Stites & Terr eds., 7^{th} ed. 1991); U.S. Pat. Nos. 4,366,241; 4,376,110; 4,517,288; and 4,837,168); Methods in Cell Biology: Antibodies in Cell Biology, volume 37 (Asai, ed. 1993).

d) Modification of Antibodies

Once an antibody of appropriate specificity and affinity has been generated the antibody can be directed conjugated to a heterologous antibody (i.e., via their respective Fc portions) to generate a bispecific binding molecule, or the antibodies can be modified prior to conjugation. Suitable modifications of the antibodies include, e.g., generation of antibody fragments or humanization of the antibodies.

Suitable antibody fragments are antibody fragments capable of specifically binding to the cardiac antigen or stem cell antigen and include, e.g., F(ab′)₂, Fab, Fv, single chain Fv (scFv), dsFv, complementarity determining regions (CDRs), V_{L} and V_{H} (see, e.g., Fundamental Immunology (Paul ed., 4d ed. 1999); Bird, et al., Science 242:423 (1988); and Huston, et al., Proc. Natl. Acad. Sci. USA 85:5879 (1988)). The antibody fragments can be obtained by a variety of methods, including, for example, digestion of an intact antibody with an enzyme, such as pepsin (to generate (Fab′)₂ fragments) or papain (to generate Fab fragments); or de novo synthesis. Antibody fragments can also be synthesized using recombinant DNA methodology. In a preferred embodiment F(ab′)₂ fragments, e.g., F(ab′)₂ fragments that specifically bind myosin light chain, troponin I, or CD45 are generated.

As mentioned above, humanized antibodies can also be generated for use in the bispecific binding molecules described herein. Humanized antibodies are antibodies in which the antigen binding loops, i.e., CDRs, comprised by the V_{H} and V_{L} regions of a non-human antibody are grafted to a human framework sequence are generated. A “humanized anti-CD45 antibody,” a “humanized anti-myosin light chain antibody” or a “humanized anti-troponin I antibody” refers to an antibody in which the antigen binding loops, i.e., CDRs, comprised by the V_{H} and V_{L} regions are grafted to a human framework sequence. Methods for humanizing or primatizing non-human antibodies are well known in the art. Generally, a humanized antibody has one or more amino acid residues introduced into it from a source which is non-human. Humanization, i.e., substitution of rodent CDRs or CDR sequences for the corresponding sequences of a human antibody, can be performed following the methods described in, e.g., U.S. Pat. Nos. 5,545,807; 5,545,806; 5,569,825; 5,625,126; 5,633,425; 5,661,016, Jones et al., Nature 321:522-525 (1986); Riechmann et al., Nature 332:323-327 (1988); Verhoeyen et al., Science 239:1534-1536 (1988); Presta, Curr. Op. Struct. Biol. 2:593-596 (1992); Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995). Transgenic mice, or other organisms such as other mammals, may also be used to express humanized or human antibodies.

2. Antibody Mimetics

In some embodiments, antibody mimetics are generated. As defined herein, antibody mimetics are protein scaffolds comprising one or more regions (i.e., loop regions) which are amenable to specific or random sequence variation such that the antibody mimetic specifically binds to an antigen of interest (e.g., a cardiac antigen or a stem cell antigen) Suitable antibody mimetics include, e.g., anticalins as described in Weiss and Lowman, Chem. Biol., 7(8)R177-84 (2000); Skerra, J. Biotechnol. 74(4):257-75; and WO 99/16873; polypeptides with a fibronectin type III domain as described in, e.g. WO 01/64942 and polypeptides with a P sandwich structure as described in, e.g., WO 00/60070.

Antibody mimetics can be generated following analysis of the antibodies described herein. The antibodies that specifically bind cardiac antigens or stem cell antigens are analyzed to identify the specific residues that are critical for antigen binding using methods known in the art including, e.g., three-dimensional crystal structure analysis of the antibody-antigen interaction. Once these residues have been identified, the loop regions of the antibody mimetics can be subjected to site directed mutagenesis such that the loop forms a binding pocket for the cardiac antigen or the stem cell antigen. Such modifications are described in, e.g., Vogt and Skerra, Chembiochem. 5(2):191-9 (2004).

The binding affinity and binding specificity of the antibody mimetics can be assayed using the binding assays known in the art and described in, e.g., Weiss and Lowman, supra and Beste et al., PNAS USA 96(5):1898-1903 (1999) which disclose immunoassays using labeled target antigens to assess the binding affinity and binding specificity of the antibody mimetics.

B. Conjugation of Binding Components

Once the antibodies, antibody mimetics, or fragments thereof that comprise binding components that specifically bind the cardiac antigen and stem cell antigen have been generated, they can be conjugated to generate the bispecific binding molecules described herein. The conjugated antibodies, antibody mimetics, or conjugated antibody fragments and can be used therapeutically as to treat cardiovascular disease (i.e., by targeting stem cells to injured myocardium). In a preferred embodiment, a (Fab′)₂ fragment that specifically binds a cardiac antigen (e.g., myosin light chain or troponin I) is conjugated to a (Fab′)₂ fragment that specifically binds a stem cells antigen (e.g., CD45).

Methods of linking two heterologous polypeptides to each other are well known in the art. In some embodiments, the antibodies or antibody fragments can be linked using chemical conjugation. In other embodiments, the antibodies or antibody mimetics can be linked using routine techniques in the field of recombinant genetics.

Methods for chemically conjugating two heterologous polypeptides (e.g., two antibodies or two antibody mimetics, or a combination thereof) are well known in the art. For example, Sen et al., J Hemathotherapy & Stem Cell Res. 10:247-260 (2001) discloses conjugation of antibodies using 2-iminothiolane HCL (Traut's reagent) and sulphosuccinimidyl 4-(N-maleimidomethyl cyclohexane-1-carboxylate. In addition, the polypeptides can be linked using a coupling reagent such as, e.g., a carbodiimide, maleimidobenzoyl-N-hydroxysuccinimide ester (MBS), or beta-maleimidopropionic acid N-hydroxysuccinimide ester (MPS), or succinic anhydride. Other methods of linking two polypeptides include, e.g., direct covalent fusion or cross-linking with glutaraldehyde. In a preferred embodiment, a (Fab′)₂ fragment that specifically binds to a cardiac antigen is conjugated to a (Fab′)₂ fragment that specifically binds to the stem cell antigen CD45. In some embodiments, intact antibodies can be conjugated to each other, e.g., via their Fe domains.

Methods for linking two heterologous polypeptides recombinantly (e.g., two antibodies; two antibody fragments, including two scFv; or two antibody mimetics; or a combination thereof) are well known in the art and are disclosed in, e.g., Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994); Bruenke et al., Br. J. Haematol. 125(2): 167-79 (2004); Kipriyanov et al., J. Mol. Biol. 330(1):99-111 (2003); Kriangkum et al., Biomol. Eng. 2001 September; 18(2):31-40 (2001); Todorovska et al.; J. Immunol. Methods 2001 Feb. 1; 248(1-2):47-66 (2001); and Pluckthun and Pack, Immunotechnology 3(2):83-105 (1997); e.g., Kostelny et al., J. Immunol. 148:1547 (1992); Pack and Pluckthun, Biochemistry 31:1579 (1992); Zhu et al., Protein Sci. 6:781 (1997); Hu et al., Cancer Res. 56:3055 (1996), Adans et al., Cancer Res. 53:4026 (1993); and McCartney, et al., Protein Eng. 8:301 (1995)).

C. Arming Stem Cells with a Bispecific Binding Molecule

Once the bispecific binding molecule has been generated, it can be bound to a stem cell. According to the methods of the invention, the bispecific binding molecule is bound to a stem cell via the stem cell antigen-specific binding component. Such a stem cell is referred to as an “armed stem cell.” In a preferred embodiment, the stem cell is armed with a bispecific antibody that specifically binds myosin light chain or troponin I; and CD45.

The stem cell may be isolated from any source known in the art and include, e.g., peripheral blood stem cells (PBSC), stem cells isolated from bone marrow; stem cells isolated from adipose tissue; mesenchymal stem cells, embryonic stem cells, CD34⁺ cells, CD34⁻ cells, CD45⁺ cells, or combinations thereof). Stem cells which express one or more of the following antigens are particularly preferred: CD9, CD29, CD34, CD44, CD45, CD49e, CD54, CD71, CD90, CD105, CD106, CD120a, CD124, CD166, Sea-1, SH2, or SH3. Exemplary stem cells and methods of isolating them are described in, e.g., Fickert et al., Osteoarthritis Cartilage 11(11):790-800 (2003) which discloses identification, quantification and isolation of human mesenchymal progenitor cells from osteoarthritic synovium; Meirelles Lda et al., Br J Haematol. 2003 November; 123(4):702-11 (2003); which discloses isolation, in vitro expansion, and characterization of mesenchymal stem cell from bone marrow; Pittenger et al., Science, Vol 284(5411): 143-147 (1999) which discloses isolation, analysis, and differentiation of adult human mesenchymal stem cells from bone marrow; and Lataillade et al., Blood 95(3):756-68 (2000) or Handgretinger et al., Bone Marrow Transplant 27(8):777-83 (2001) which disclose isolation, analysis, and purification of adult human peripheral blood CD34⁺ progenitor cells; U.S. Pat. No. 6,667,034 which discloses isolation and differentiation of stem cells from human hematopoietic cells, i.e., from bone marrow and peripheral blood; and U.S. Pat. No. 6,261,549 which discloses isolation of human mesenchymal stem cells from peripheral blood; and Gepstein, Circ. Res. 91(10):866-76 (2002) which discloses derivation of embryonic stem cells.

Typically, stem cells are purified from peripheral blood using methods known in the art including, e.g., immunomagnetic selection with the MACS system (Miltenyi Biotech, Tebu) or antibody-coated Dynabeads (Dynal Biotech, Oslo). Typically a heterogeous population of cells is contacted with antibody-coated magnetic beads. The antibody specifically binds to a cell surface marker differentially or preferentially expressed on the surface of a stem cell, thereby forming a complex between the beads and the stem cells in the heterogenous population. The labeled stem cells can then be isolated from the heterogenous cell population using methods known in the art including, e.g., flow cytometry.

In some embodiments, the stem cells are primed, i.e., treated with a cytokine or a mixture of cytokines, or transformed with genes for somatic gene delivery (see, e.g., (Ausubel et al., 1994, supra) prior to being armed with the bispecific binding molecules described herein. Priming can facilitate homing of the stem cell to the injured cardiac tissue and differentiation or transdifferentiation of the stem cell after it has homed to the injured cardiac tissue. For example, bone marrow derived stem cells are typically primed with G-CSF to facilitate their homing from the bone marrow to the peripheral blood prior to being armed with bispecific antibody that specifically binds to myosin light chain and CD45.

Once isolated, the stem cells can be administered to a patient directly or can be armed with the bispecific binding molecules described herein prior to administration to a patient. Often, arming the stems cells comprises incubating the stem cells with a bispecific antibody under conditions such that the bispecific antibody binds to the stem cell to create a bispecific antibody-stem cell complex. One of skill in the art will appreciate that suitable ratios of bispecific antibody: stem cell can be selected based on the particular properties of the bispecific antibody and the stem cell. Typically about 0.05 to 500 ng, about 5 to about 400 ng, about 10 to about 300 ng, about 25 to about 250 ng, about 40 to about 100 ng, or about 50 ng of bispecific antibody per 10⁶ stem cells is sufficient to generate a population of bispecific antibody-stem cell complexes suitable for use in the methods of the invention.

### III. METHODS OF THE INVENTION

One embodiment of the present invention provides a method of targeting stem cells to injured cardiac tissues. Stem cells are “armed” with the bispecific polypeptides described herein are administered to a patient with an injured myocardium (i.e., a patient with a cardiovascular disorder. The stem cells may be autologous to the patient with the cardiovascular disorder, or may be obtained from an allogeneic donor. The pharmaceutical composition described herein can conveniently be used to deliver the “armed” stem cells described herein.

The present invention also relates to a pharmaceutical composition comprising the bispecific antibodies in a pharmaceutically acceptable carrier. In some embodiments, the pharmaceutical compositions comprise stem cells armed with the bispecific antibodies described herein. In therapeutic applications, compositions are administered to a patient suffering from a disease (e.g., cardiovascular disease), in an amount sufficient to cure or at least partially arrest the disease and its complications, i.e., by repairing injured myocardium. An amount adequate to accomplish this is defined as a therapeutically effective dose. Amounts effective for this use will depend on the severity of the cardiovascular disease and the general state of the patient's health.

The pharmaceutical compositions of the present invention (i.e., compositions comprising bispecific antibodies or armed stem cells) may be administered by any means known in the art. Preferably, the compositions are suitable for parenteral administration (e.g., intravenous, intraperitoneal). The compositions of the invention may also be administered subcutaneously, into vascular spaces, or into joints, e.g., intraarticular injection.

Single or multiple administrations of the compositions may be administered depending on the dosage and frequency as required and tolerated by the patient. In any event, the composition should provide a sufficient quantity of the armed stem cells to effectively treat the patient, i.e., to repair or augment repair of injured myocardium.

Preferably, the compositions for administration comprise a solution of the composition and a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, sterilization techniques known in the art. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The composition comprising armed stem cells may also formulated in microspheres, liposomes or other microparticulate delivery systems. The concentration of composition comprising armed stem cells in these formulations can vary widely, and will be selected primarily based on fluid volumes, viscosities, body weight and the like in accordance with the particular mode of administration selected and the patient's needs.

Thus, typical pharmaceutical composition comprising bispecific antibodies for intravenous administration would be about 0.05 to 500 ng, about 5 to about 400 ng, about 10 to about 300 ng, about 25 to about 250 ng, about 40 to about 100 ng, or about 50 ng of bispecific antibody per patient per day. A typical pharmaceutical composition comprising armed stem cells for intravenous administration would be about 10⁵ to about 4×10⁶ cells, about 5×10⁵ about 3×10⁶ cells, or about 10⁶ to about 2.5×10⁶ cells, or about 1.5×10⁶ to about 2.0×10⁶ cells per patient per day. Methods for preparing parenterally administrable compositions will be known or apparent to those skilled in the art and are described in more detail in such publications as Remington's Pharmaceutical Science, 17th Ed., Mack Publishing Co., Easton, Pa., (1985).

Typically, the pharmaceutical compositions comprising armed stem cells are administered in a therapeutically effective dose over either a single day or several days by daily intravenous infusion. The dose will be dependent upon the properties of the composition comprising armed stem cells employed, e.g., its activity and biological half-life, the concentration of the composition comprising armed stem cells in the formulation, the site and rate of dosage, the clinical tolerance of the patient involved, the extent of cardiovascular disease afflicting the patient and the like as is well within the skill of the physician.

The compositions may be administered in solution. The pH of the solution should be in the range of pH 5 to 9.5, preferably pH 6.5 to 7.5. The compositions thereof should be in a solution having a suitable pharmaceutically acceptable buffer such as phosphate, tris (hydroxymethyl) aminomethane-HCl or citrate and the like. Buffer concentrations should be in the range of 1 to 100 mM. The solution of the compositions may also contain a salt, such as sodium chloride or potassium chloride in a concentration of 50 to 150 mM. An effective amount of a stabilizing agent such as albumin, a globulin, a detergent, a gelatin, a protamine or a salt of protamine may also be included and may be added to a solution containing the immunoconjugate or to the composition from which the solution is prepared. In some embodiments, systemic administration of the composition comprising armed stem cells is typically made every two to three days or once a week if a humanized form of the antibody is used. Alternatively, daily administration is useful. Usually administration is by intravascular infusion.

The compositions described herein (i.e., bispecific antibodies or armed stem cells) can be administered to a patient in conjunction with other therapies for cardiovascular disease. For example, the compositions can be administered in conjunction with angioplasty to promote repair of injured cardiac tissue. The compositions can be administered prior to the angioplasty, contemporaneous with the angioplasty, or subsequent to the angioplasty.

### EXAMPLES

### Example 1

### Materials and Methods

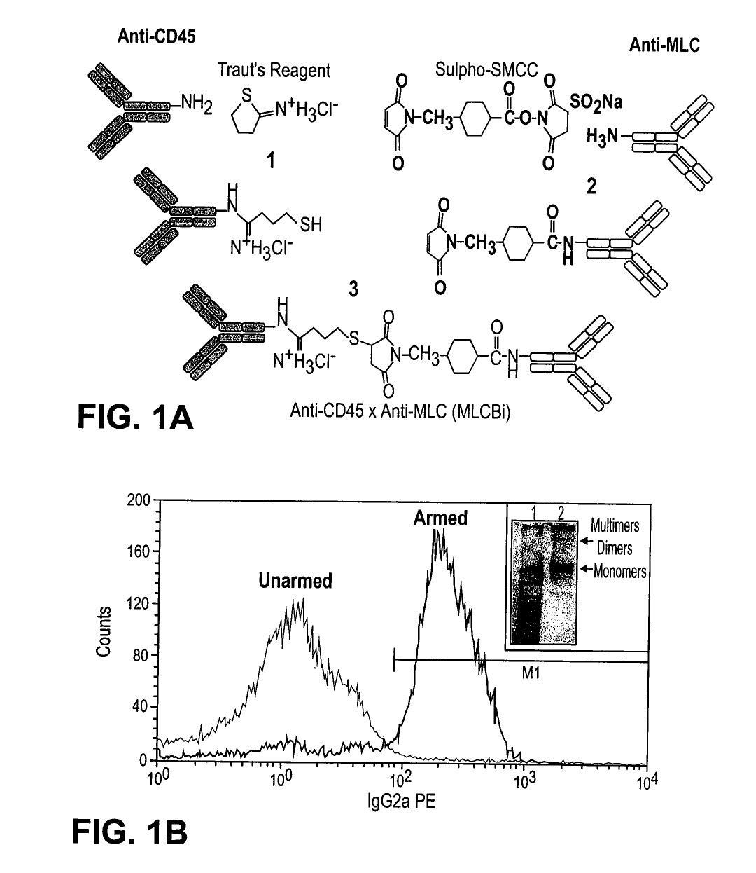

Conjugation and Analysis of anti-CD45 x anti-MLC BiAb: An anti-myosin LC (“anti-MLC”) monoclonal antibody and an anti-CD45 monoclonal antibody (“anti-CD45”) were generated using methods known the in the art (see, e.g. Harlow and Lane, supra). The anti-MLC antibody was heteroconjugated to the anti-CD45 antibody to produce a bispecific antibody (“BiAb”), anti-CD45 x anti-MLC (FIG. 1). Briefly, anti-CD45 (1-5 mg) in 50 mM NaCl, 1 mM EDTA, pH 8.0 was reacted with a 5-10 fold M excess of Traut's reagent (2-iminothiolane HC1, Pierce) and anti-MLC (1-5 mg) in 0.1 M sodium phosphate, 0.15 M NaCl at pH 7.2 was reacted with a 4-fold molar excess of sulphosuccinimidyl 4-(N-maleimidomethyl)cyclohexane-1-carboxylate (Sulpho-SMCC) at room temperature for 1 hr. Both antibodies were then purified on PD-10 columns in PBS to remove unbound cross-linker. The cross-linked mAbs were mixed immediately at equimolar ratios and conjugated at 4° C. overnight.

Anti-human CD45 (BD Pharmigan, San Diego, Calif.) was cross-linked with Traut's reagent and anti-MLC (Abcam, Cambridge, Mass.) was cross-linked with SulfoSMCC as described (Sen et al., Journal of Hematotherapy & Stem Cell Research, 10:247-260 (2001)). The cross-linked antibodies were mixed and allowed to heteroconjugate overnight to produce anti-CD45 x anti-MLC (CD45xMLC). The proportion of dimers, multimers, and monomers were determined by non-reducing SDS-PAGE and the concentration of the entire mix determined as described. The arming dose of CD45xMLC was 50 ng/million PBMC or purified CD34⁺ cells.

Cell purification: Unused, excess G-CSF primed PBMC were obtained by leukopheresing normal human stem cell donors. All blood collection and use of human blood products for research were conducted under Internal Review Board approved protocols at Roger Williams Hospital and signed consents were obtained from the donors. CD34+ purified cells were obtained by positive selection over a clinical Isolex 300i column and the CD34+ selection kit (Baxter, Deerfield, Ill.). The G-CSF primed PBMC or purified CD34+ cells were frozen in convenient aliquots and thawed, washed, and armed with CD45xMLC and infused within 24 hours of arming.

Myocardial injury model: A previously described ischemia reperfusion model was used in this study (Siever et al., Magnetic Resonance in Medicine, 10:172-181 (1989); Christman et al., Tissue Eng., 10:403-409 (2004)). Nude rats (225-250 g) were endotracheally intubated, ventilated with a rodent ventilator (Harvard Apparatus) and anesthetized with inhalational isofluorane. A 7-0 Ticron suture was placed around the left anterior descending portion (LAD) of the left coronary artery. The suture was tightened to occlude the LAD for 17 minutes and then removed to allow for reperfusion. The sternotomy was then closed and the animal was allowed to recover. Experience with this model has previously demonstrated that this technique results in an acute infarct size of at least 30% of the left ventricle (LV) with reperfusion (Wolfe et al., Circulation, 80:969-982 (1989); Zhu, B. et al., J. Am. Coll. Cardiol., 35:787-795 (2000); Zhu et al., J. Renin. Angiotensin. Aldosterone. Syst., 2:129-133 (2001)).

Intravenous administration of hematopoietic stem cells: Two days after myocardial infarction, either CD45xMLC-armed PBMC (8×10⁶, n=12), unarmed PMBC (8×10⁶, n=12), CD45xMLC-armed CD34⁺ cells (2×10⁶, n=9) or unarmed CD34⁺ cells (2×10⁶, n=8) were injected intravenously via the right internal jugular vein. Animals receiving armed and unarmed PBMC were sacrificed either 24 hours or at 5 weeks after the injection of cells. Animals receiving armed and unarmed CD34⁺ cells were sacrificed 5 weeks after the injection of cells.

Echocardiography: Transthoracic echocardiography was performed on all animals receiving CD34⁺ cells in the conscious state prior to MI; and 12 days and 35 days after the MI. The study was concluded 5 weeks following infarction at which point the remodeling process in the rat is essentially complete (Fishbein et al., Am J Pathol, 90:57, (1978)). The methodology of echocardiography used in this study has been previously described (Doursout et al., Ultrasound Med. Biol., 27:195-202 (2001)) and other reports have demonstrated the accuracy and reproducibility of transthoracic echocardiography in rats with myocardial infarcts (Youn et al., J Am Soc Echocardiogr, 12:70 (1999); Nakamura et al., Am J Physiol Heart Circ Physiol., 281:H1104 (2001); Litwin et al., Circulation, 89:345 (1994)).

Histology: Either 24 hours or 5 weeks following the injection of cells, rats were euthanized with a pentobarbital overdose (200 mg/kg). The hearts were perfused with saline, rapidly excised, fresh frozen in Tissue Tek O.C.T. freezing medium (Sakura) and sectioned into 5 μm slices, equally distributed throughout the infarct area as previously described (Christman et al., J. Am. Coll. Cardiol., 44:654-660 (2004)).

Immunohistochemical staining was blinded and performed according to the manufacturer using the Animal Research Kit (ARK; DakoCytomation, Carpinteria, Calif.) combined with tyramide amplification (Dako CSA System) following high pH Target Retrieval and endogenous peroxidase quenching (DakoCytomation). The mouse BiMAb CD45 x MLC was detected in cardiac tissue by goat anti-mouse Ig (CSA Peroxidase System, DakoCytomation, Capinteria, Calif.). Anti-human HLA-DR (MHC II) (1.5 μg/ml) and anti-human HLA-A,B,C (MHC I) (3 ug/ml) (BD Biosciences) were reacted with biotinylating and blocking reagents (ARK System) just prior to incubation with sections for 1 hour at room temperature. Primary antibodies were detected with streptavidin-biotin complex, and the signal was amplified and visualized by diaminobenzidine precipitation at the antigen site (CSA System). Samples were counterstained with hematoxylin. Adjacent normal myocardium or sections from rats treated with unarmed cells served as IHC controls. Immunohistochemically stained cells within the region of the infarct were manually quantified after using the color select function in Adobe Photoshop 5.5 to highlight individual cells stained brown with the diaminobenzidine substrate.

Immunoflourescence staining was performed five weeks after the intravenous administration of cells from the following groups: 1) armed PBMC, 2) unarmed PBMC, 3) armed CD 34+ cells and 4) unarmed CD34+ cells. Five sections equally distributed through the infarct area were double labeled with mouse anti-human HLA (BD bioscience, 1:200 dilution) and either rabbit anti-smooth muscle alpha actin (Lab Vision, 1:300 dilution), rabbit anti-Troponin T (Abcam, 1:300 dilution), or rabbit anti-Cx43 (Sigma, 1:300 dilution). In order to visualize labeled cells, slides were incubated with secondary antibodies anti-mouse Alexa 488 (molecular probe, 1:500 dilution) and anti-rabbit Rhodamine (molecular probe, 1:100 dilution).

Statistics: Statistical analyses were performed using GraphPad Prism version 4.00 for Windows (GraphPad Software, San Diego, Calif.). Results from cell density measurements were compared using the unpaired t-test. Data is presented as mean±standard deviation. Echocardiography measurements were compared using a student's t-test.

### Example 2

### Targeted G-CSF Primed PBMC Specifically Localize to Infarcted Regions of Rat Myocardium

The BiAb anti-CD45 x anti-MLC (CD45xMLC) was produced by chemical heteroconjugation as shown in FIG. 1A and as described in the methods (Sen et al., Journal of Hematotherapy & Stem Cell Research, 10:247-260 (2001)). The typical preparation consists of 12% conjugated dimers, 66% unconjugated monomers, and 22% multimers, as shown by Western blot (FIG. 1B, inset). Binding of the BiAb to PBMC via its anti-CD45 moiety was demonstrated using a goat anti-mouse IgG2a PE-conjugated antibody that recognized the mouse IgG2a anti-MLC arm of the BiAb. Cryopreserved GCSF primed PBSC were thawed, armed with 50 ng of anti-CD45 x anti-rat anti-myosin LC per million PBSC (FIG. 1B shows the results of flow cytometric analyses of a sample from the PBSC either armed or unarmed with anti-CD45 x anti-myosin LC), and injected intravenously into rats 48 hrs after transient ligation of the LAD artery.

The ability of CD45xMLC to localize to the site of myocardial injury via its anti-MLC moiety was determined. Anti-CD45 x anti-myosin LC BiMab armed G-CSF primed PBSC (8×10⁶) or unarmed PBSC alone were infused intravenously into nude rats 48 h post-ligation of the left anterior descending artery (LAD) followed by reperfusion. Rats were euthanized 24 hours after treatment with the PBSC and hearts were snap-frozen, sectioned and fixed with paraformaldehyde. Mouse anti-CD45 x anti-myosin LC BiMAb was detected in cardiac tissue at the site of infarction by IHC with ready-to-use biotinylated goat anti-mouse IgG2a (CSA Peroxidase System, DakoCytomation, Capinteria, Calif.) after target retrieval and endogenous peroxidase quenching with the CSA Ancillary System (DakoCytomation). The signal was amplified and visualized by diaminobenzidine precipitation at the antigen site. Samples were counterstained with hematoxylin. The average (mean±SD) positive pixels/high power field (hpf) in the infracted area was 5173±1411 in rats that had received armed PBMC and 461±40 for staining in the infracted area of rats that received unarmed PBMC (i.e. no CD45xMLC). Non-infarcted areas were not significantly different than background.

### Example 3

### Arming with CD45xMLC Enhances Homing and Persistence of Human G-CSF Primed PBMC at the Site of Myocardial Infarctions

Cryopreserved G-CSF primed PBMC were thawed, armed with 50 ng of bispecific antihuman CD45 x anti-rat myosin light chain (MLC) antibody per million PBMC, and injected (8×10⁶ cells) intravenously into nude rats 48 hrs after 17 minute ligation of the LAD artery followed by reperfusion. Rats were euthanized 5 weeks after cellular treatment and hearts were snap-frozen, sectioned and fixed with paraformaldehyde. Numerous human cells were detected in cardiac tissue at the site of infarction following immunohistochemical staining according to the manufacturer's instructions using the Animal Research Kit (ARK; DakoCytomation, Carpinteria, Calif.) combined with tyramide amplification (Dako CSA System) following high pH Target Retrieval and endogenous peroxidase quenching (DakoCytomation). Anti-HLA-DR (1.5 μ/ml) and anti-HLA-A,B,C (3 μ/ml) (BD Biosciences) were reacted with biotinylating and blocking reagents (ARK System) just prior to incubation with sections for 1 h at room temperature. Primary antibodies were detected with streptavidin-biotin complex, and the signal was amplified and visualized by diaminobenzidine precipitation at the antigen site (CSA System). Samples were counterstained with hematoxylin. As controls, staining of adjacent normal myocardium (same section) as well as stained sections from rats treated with unarmed PBMC. These results confirmed that “armed” PBMC can be specifically directed with an injury-specific cardiac muscle adhesion molecule.

To demonstrate the efficacy of CD45xMLC to traffic PBMC to the site of myocardial injury, cardiac sections were obtained 24 hours after infusions of armed PBMC. The sections were stained for human MHC class I (HLA-I) or human MHC class II (HLA-II) antigens. In rats infused with CD45xMLC-armed PBMC, large numbers of HLA-I positive and HLA-II positive cells localized to the MIs, indicating the presence of human leukocytes and stem cells. Very few cells of either population were present in the MIs of rats administered unarmed PBMC. At 5 weeks post-infusion, more human cells were found to persist in CD45xMLC-armed PBMC-treated rats with a predominance of HLA-I positive cells over HLA-II positive cells in the MIs. Importantly, the morphology of these HLA-I positive cells appeared to be non-hematopoietic. Consistent with the disappearance of HLA-II positive cells, staining for anti-human CD45 or anti-human CD3 was negative, confirming the absence of human leukocytes or T cells. Immunohistochemical staining of the area of injured myocardium demonstrated several cells with co-localization of HLA class I and troponin T labeling in CD45xMLC armed PBMC, while myocardium of rats receiving unarmed PBMC showed only a rare cell with co-localization of HLA class I and troponin T labels.

### Example 4

### CD45xMLC-Armed CD34+ Cells Persist in MIs and Co-Express Human Class I and Muscle Specific Antigens 5 Weeks after Infusion

Based on the persistence of HLA-I positive cells in the infarcted region after targeting CD45xMLC-armed PBMC, we enriched for CD34+ cells from G-CSF primed normal PBMC to test whether CD34+ cells could be targeted to MIs and preserve left ventricular function. Cryopreserved G-CSF primed PBMC were thawed, armed with 50 ng of antihuman CD45 x anti-rat myosin light chain (MLC) per million PBMC, and injected (8×10⁶ cells) intravenously into nude rats 48 hrs after 17 minute ligation of the LAD artery followed by reperfusion. By flow cytometric phenotyping, CD34+ cells comprised 0.5% of the PBMC population. Isolex 300i-purified CD34+ cells contained 99% CD34+, 99% CD45+, 99% CD 38+, 96% CD117+, 87% CD 133+ and 70% CD33+ cells by flow cytometry. Flow cytometry also showed that the CD34+ cells were negative for CD4, CD7, CD10, CD16, CD19, CD20 and CD23 antigens. Two days after myocardial infarctions, rats received either 2×10⁶ CD45xMLC-armed CD34+ cells or unarmed CD34+ cells intravenously.

Rats were euthanized 5 weeks after treatment and hearts were snap-frozen, sectioned and fixed with paraformaldehyde and staining for human cells in cardiac tissue at the site of infarction was conducted. Five weeks after the infusion of CD45xMLC-armed CD34+ cells, HLA-I positive cells significantly outnumbered HLA-II positive cells in the infarcted region (171.8±52.7 MHC class I+cells/hpf vs. 58.5±13.1 MHC class II+cells/hpf, p<0.006). The number of persistent Class II positive cells in the MIs of rats that received BiAbCD45/MLC-armed PBMC was similar to background levels of 45.3±4.9 Class II+ cells/hpf and comparable to numbers seen in rats that received 8×10⁶ unarmed cells (25.9±1.7 MHC class II+ cells/hpf). Human cells bearing HLA markers were distributed throughout the infarct region. There was a much higher concentration of HLA-I positive cells at the borderzone of the infarction. Two-color staining for human HLA-I and rat troponin T consistently found double stained cells in infarcts of rats treated with the CD45xMLC-armed CD34+ cells. This finding suggests that muscle antigen-expressing cells that co-stained for HLA-I developed from the CD45xMLC-targeted CD34+ cells within 5 weeks of the infusion. The infarcts of rats that received unarmed CD34+ cells showed only rare human HLA-I and rat troponin T double positive cells. However, the double stained HLA-I and rat troponin T positive cells in the armed group, comprised only a small fraction of the total cells staining for HLA-I.

Co-localization of staining for rat α-smooth muscle actin and HLA-I was also observed in MIs of rats 5 weeks after infusion of CD45xMLC-armed CD34+ cells. The localization of HLA-I positive cells to the vascular wall of arterioles suggests that the cells may contribute to new blood vessel formation.

### Example 5

### BiMab Targeted CD 34⁺ Cells to Ischemic Myocardium

To test whether peripheral CD34⁺ cells can be targeted to ischemic myocardium and prevent the negative remodeling aspects associated with a MI, we compared CD34⁺ cells armed with BiMab CD45 x anti-MLC with unarmed CD34⁺ cells. Nude rats (female, 200-225 g) underwent 17 minutes of occlusion of the left coronary artery followed by reperfusion. Two days after the MI, rats received either: 1) 2 million armed CD34⁺ cells intravenously (n=6) or 2) 2 million unarmed CD34⁺ cells intravenously (n=3). Serial echocardiograms were performed at 7-10 days post-MI and at 5 weeks post-MI. Transthoracic echocardiography in rats with myocardial infarcts have been shown to accurately and reproducibly assess left ventricular dimensions and function (Litwin et al., Circulation, 89:345-54 (1994)) and has previously been described (Doursout et al., Ultrasound Med Biol., 27:195-202 (2001)). Two-dimensional images were obtained in both parasternal long and short axis views (at the papillary muscle level). Enhanced resolution imaging function (RES) was activated with a region of interest adjusted to heart size whenever possible. Two criteria were used for adequate imaging. First, the short-axis view must demonstrate at least 80% of the endocardial and epicardial border. Second, the long-axis view must demonstrate the plane of mitral valve, where the annulus and the apex could be visualized. After adequate two-dimensional images were obtained, the M-mode cursor was positioned perpendicular to the ventricular anteroseptal wall (at the site of infarct) and the posterior wall, at the level of the papillary muscles. Wall thickness and left ventricular internal dimensions were measured according to the leading edge method of the American Society of Echocardiography. Fractional shortening (FS) as a measure of systolic function was calculated as FS (%)=[(LVIDd−LVIDs)/LVIDd]×100%, where LVID was the left ventricular internal dimension, d was diastole and s was systole. Acquisition of echocardiographic images and data analysis were performed in a blinded fashion. The animals were sacrificed five weeks after administration of treatment. Hearts were harvested, fresh frozen and histologically analyzed for MI size, wall thickness, SC transplantation and SC transdifferentiation into either vascular structures or cardiac muscle.

There was no significant difference in FS and ventricular volumes between the two groups at 7-10 days post-MI. At 5 weeks, the fractional shortening (FS) of the unarmed group decreased significantly compared to the armed group (0.19+0.03 vs 0.33+0.06; P=0.02), while LVDS of the unarmed group significantly increased as compared to the armed group (0.67+0.04 vs 0.47+0.12; p=0.04). There was a nonsignificant trend for increase wall thickness in the armed group as compared to the unarmed group. Histological analysis of MI size and wall thickness demonstrated a nonsignificant trend toward smaller MI in the armed group as compared to the unarmed group and increased wall thickness in the armed group as compared to the unarmed group. The histological trends corroborate the echo trends of the beneficial effects of targeted CD34⁺ cell therapy to decrease MI size, prevent the negative remodeling effects of a MI which leads to improved LV function. These preliminary findings will be corroborated with high resolution MRI. Immunohistochemistry studies demonstrate that armed CD34⁺ cells specifically localize to the infarct region of the heart, co-localize with cardiac cells (indentified with troponin I) and co-localize with smooth a-actin stained cells. There are rare unarmed cells to the infarct region.

These preliminary studies show that human cells can be successfully targeted to ischemic injured myocardium in nude rats and that the homing and residence of the cells is limited in adjacent normal myocardium. Additionally, our data suggests that cells homed to the ischemic myocardium can transdifferentiate. Our data corroborate studies of Yeh et al. (Yeh et al., Circulation, 108(17):2070-3 (2003)) who reported that intravenous delivery of CD34⁺ cells transdifferentiate into myocardial cells, mature endothelial cells and smooth muscle cells in vivo. Our data also demonstrate that a systemic approach for cell-based therapy is possible which would greatly simplify and increase the safety of a cellular based treatment for the reconstruction of damaged myocardium.

### Example 6

### Improved Left Ventricular Function in Rats Treated with CD45xMLC-Armed CD34⁺ Cells

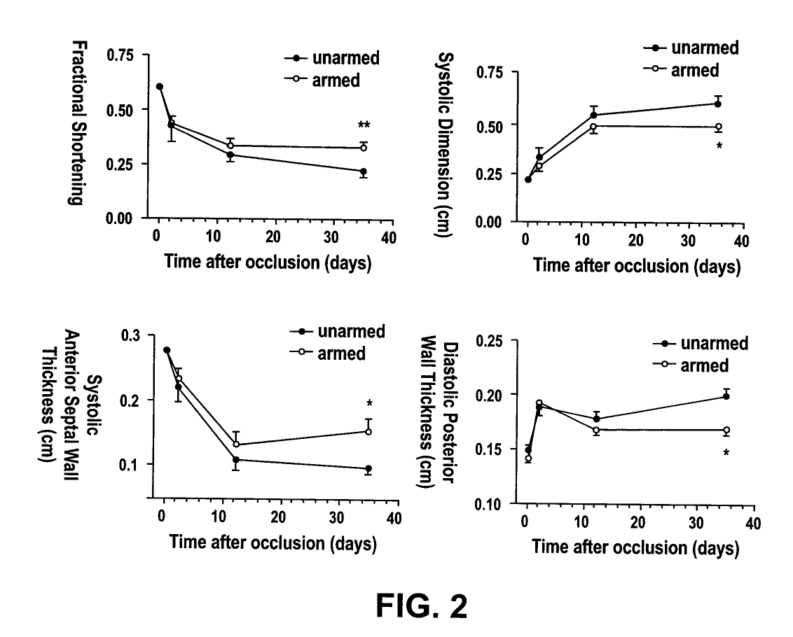

Sequential echocardiograms of rats that received CD45xMLC-armed CD34⁺ cells showed significantly better cardiac function compared to rats that received unarmed CD34+ cells (FIG. 2). By 5 weeks, the fractional shortening (FS) of the unarmed group (0.23±0.08; n=8) decreased significantly (p<0.01) compared to the CD45xMLC-armed group (0.34±0.06; n=9), while LV systolic diameter (SD) of the unarmed group (0.60±0.04) was significantly (p<0.05) dilated compared to the CD45xMLC-armed group (0.49±0.12). Systolic anterior wall thickness (AWTs) of the unarmed group (0.10±0.03) was significantly (p<0.05) thinner than the AWTs of the CD45xMLC-armed group (0.15±0.06), while the posterior wall had a compensatory increase in the diastolic posterior wall diameter thickness (PWDd) in the unarmed group as compared to the PWDd of the CD45xMLC-armed group (0.20±0.02 vs 0.17±0.02; p<0.05).

It is understood that the examples and embodiments described herein are for illustrative purposes only and that various modifications or changes in light thereof will be suggested to persons skilled in the art and are to included within the spirit and purview of this application and are considered within the scope of the appended claims. All publications, patents, patent applications, and Accession Nos. cited herein are hereby incorporated by reference in their entirety for all purposes.

What is claimed is:

1. A composition, the composition comprising a polypeptide comprising a cardiac antigen-specific binding component and a stem cell antigen-specific binding component,
wherein the cardiac antigen-specific binding component comprises a polypeptide that specifically binds to a cardiac antigen available for binding to the cardiac antigen-specific binding component, wherein the cardiac antigen is selected from the group consisting of: myosin light chain and troponin I; and
wherein the stem cell antigen-specific component comprises a polypeptide that specifically binds to an antigen expressed on the surface of a stem cell.

2. The composition of claim 1, wherein the cardiac antigen-specific binding component and the stem cell antigen-specific binding component are chemically conjugated.

3. The composition of claim 1, further comprising the stem cell bound to the stem cell antigen-specific binding component.

4. The composition of claim 3, wherein the stem cell is selected from the group consisting of: a peripheral blood stem cell (PBSC), a stem cell isolated from bone marrow; a stem cell isolated from adipose tissue; a mesenchymal stem cell, an embryonic stem cell, an umbilical cord blood stem cell, a CD34⁺ cell, a CD34⁻ cell, a CD45⁺ cell, and combinations thereof.

5. The composition of claim 4, wherein the stem cell is a PBSC.

6. The composition of claim 1, wherein the cardiac antigen-specific binding component and the stem cell antigen-specific binding component are each antibodies.

7. The composition of claim 6, wherein the antibodies are (Fab)′₂ fragments.

8. The composition of claim 6, wherein the antibodies are scFv.

9. The composition of claim 6, wherein the antibodies are humanized.

10. The composition of claim 6, wherein the stem cell antigen-specific binding component is an antibody that specifically binds to a member selected from the group consisting of: CD9, CD29, CD34, CD44, CD45, CD49e, CD54, CD71, CD90, CD105, CD106, CD120a, CD124, CD166, Sca-1, SH2, SH3, and HLA Class I.

11. The composition of claim 10, wherein the stem cell antigen-specific binding component is an antibody that specifically binds to CD45.

12. A method for targeting stem cells to injured cardiac tissue, the method comprising:
administering a composition to a subject, said composition comprising a polypeptide comprising a cardiac antigen-specific binding component and a stem cell antigen-specific binding component,
wherein the cardiac antigen-specific binding component comprises a polypeptide that specifically binds to a cardiac antigen available for binding to the first component, wherein the cardiac specific antigen is selected from the group consisting of: myosin light chain and troponin I;
wherein the stem cell antigen-specific binding component comprises a polypeptide that specifically binds to an antigen expressed on the surface of a stem cell; and
wherein the polypeptide is bound to the stem cell.

13. The method of claim 12, wherein the first binding component and the stem cell antigen-specific binding component are chemically conjugated.

14. The method of claim 12, wherein the stem cell is a peripheral blood stem cell.

15. The method of claim 12, wherein the cardiac antigen-specific binding component and the stem cell antigen-specific binding component are each antibodies.

16. The method of claim 15, wherein the antibodies are (Fab)′2 fragments.

17. The method of claim 15, wherein the antibodies are scFv.

18. The method of claim 15, wherein the antibodies are humanized.

19. The method of claim 15, wherein the second binding component is an antibody that specifically binds to CD45.

20. The method of claim 12, wherein the subject is a mammal.

21. The method of claim 20, wherein the mammal is a human.

22. Use, in the manufacture of a medicament for targeting stem cells to injured cardiac tissue of a polypeptide comprising a cardiac antigen-specific binding component and a stem cell antigen-specific binding component,
wherein the cardiac antigen-specific binding component comprises a polypeptide that specifically binds to a cardiac-specific antigen available for binding to the first component, wherein the cardiac specific antigen is selected from the group consisting of: myosin light chain and troponin I; and
wherein the stem cell antigen-specific binding component comprises a polypeptide that specifically binds to an antigen expressed on the surface of a stem cell.

23. The use of claim 22, wherein the polypeptide is bound to the stem cell.

24. The use of claim 23, wherein the stem cell is selected from the group consisting of: a peripheral blood stem cell (PBSC), a stem cell isolated from bone marrow; a stem cell isolated from adipose tissue; a mesenchymal stem cell, an embryonic stem cell, an umbilical cord blood stem cell, a CD34⁺ cell, a CD34⁻ cell, a CD45⁺ cell, and combinations thereof.

25. The use of claim 23, wherein the antigen expressed on the surface of a stem cell is selected from the group consisting of: CD9, CD29, CD34, CD44, CD45, CD49e, CD54, CD71, CD90, CD105, CD106, CD120a, CD124, CD166, Sca-1, SH2, SH3, and HLA Class I.
